(19) United States
(12) Patent Application Publication
(10) Pub. No.: US 2008/0153019 A1
(43) Pub. Date: Jun. 26, 2008

(54) ARYLAMINE COMPOUND AND ELECTROPHOTOGRAPHIC PHOTORECEPTOR

(75) Inventors: Yahui WANG, Dalian (CN); Tetsuya TOMINAGA, Hiratsuka-shi (JP); Shinji KUBO, Hiratsuka-shi (JP)

Correspondence Address: SUGHRUE-265550, 2100 PENNSYLVANIA AVE. NW, WASHINGTON, DC, 20037-3213, US

(73) Assignee: FUJIFILM FINECHEMICALS CO., LTD., Tokyo (JP)

(21) Appl. No.: 11/964,372

(22) Filed: Dec. 26, 2007

(30) Foreign Application Priority Data
Dec. 26, 2006 (JP) 2006-349861

(51) Int. Cl. G03C 1/73; C07C 211/54

(52) U.S. Cl. 430/57.3; 564/434; 430/57.4

(57) ABSTRACT

An arylamine compound represented by formula (1):

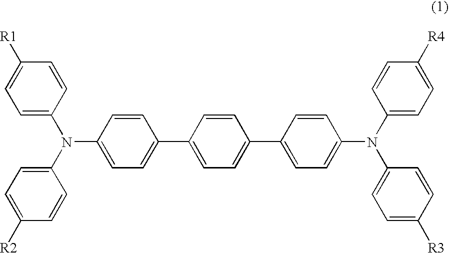

wherein R1 to R4 each independently represents a linear alkyl group or an arylalkenyl group, provided that when all of R1 to R4 represent linear alkyl groups, at least one of R1 to R4 represents a linear alkyl group having a carbon number of 3 or more; and an electrophotographic photoreceptor.

### BACKGROUND OF THE INVENTION

1. Field of the Invention

The present invention relates to a novel arylamine compound and an electrophotographic photoreceptor used for image formation by an electrophotographic system such as electrophotographic copying machine, laser beam printer and LED printer.

2. Description of the Related Art

The electrophotographic system is a kind of image forming method, where the surface of a photoreceptor generally using a photoelectrically conductive material is electrically charged in a dark place and exposed, the electric charge in the exposed area is electrically neutralized to obtain an electrostatic image, and this electrostatic image is developed using a toner and then transferred and fixed on paper or the like to obtain an image. As regards the photoreceptor used therefor, an inorganic photoelectrically conductive material such as selenium, zinc oxide, cadmium sulfide and amorphous silicon has been conventionally prevailing, but this material has many problems, for example, is sensitive to heat or mechanical impact, involves high production cost or has toxicity. For this reason, at present, an organic photoreceptor composed of various organic materials and improved in those problems is widely used in practice. As such an organic photoreceptor, there have been proposed, for example, a multilayer photoreceptor in which a charge generating layer and a charge transport layer are stacked, and a single-layer photoreceptor in which a charge generating agent and a charge transport agent are dispersed in a single photosensitive layer.

Recently, the image forming method by an electrophotographic system is propagated in a hard copy printer of personal computers, or in the case of a copying machine, a digital image forming method using LED or a laser as the exposure light source is abruptly spread. Furthermore, with the progress of a high-definition digital image, a technique of producing a high-quality high-power electrophotographic image has been developed and studies are continuing at present. For obtaining such a high-quality high-power electrophotographic image, it is necessary to combine a charge generating agent having high charge generation efficiency with a charge transport agent having high charge transport ability. The properties required of the charge transport agent having high charge transport ability are to efficiently receive the electric charge generated from a charge generating material upon light irradiation under an applied voltage, to migrate in the photosensitive layer at a high speed, and to swiftly attenuate the surface potential. The speed at which the electric charge migrates per unit electric field is called charge mobility. Higher charge mobility means that the electric charge migrates at a higher speed in the transport layer, and this leads to a high possibility of obtaining a high-quality high-power electrophotographic image. Accordingly, in order to achieve high sensitivity and high power of an electrophotographic photoreceptor, use of a material having high charge mobility is indispensable, but a satisfactory compound is not yet found out at present.

In the case of using a charge transport material by dissolving it together with a binder polymer in an organic solvent and coating the solution, it is required to form a uniform organic thin film without causing precipitation of a crystal or production of a pinhole in the formed film coating. Because, in an organic photoreceptor, when a high electric field is applied, dielectric breakdown or noise may occur in the portion where such a fine crystal or pinhole is produced. Therefore, the charge transport material is required to have excellent solubility in an organic solvent and excellent film-forming property. However, many of the compounds reported so far have various problems, for example, a crystal precipitates after film formation or even if film formation can be completed, the durability is bad, though these materials may have high charge mobility than ever.

Several organic materials for an electrophotographic photoreceptor, where a p-terphenyl compound is used, have been recently proposed (see, International Publication No. 2005/115970, JP-A-2001-305764 (the term “JP-A” as used herein means an “unexamined published Japanese patent application”), U.S. Pat. No. 4,273,846, U.S. Pat. No. 5,707,768, U.S. Pat. No. 5,840,980 and JP-A-2003-021921). The p-terphenyl compounds have excellent hole mobility, but the mobility is not yet satisfied. Also, in view of solubility in a solvent, problems to be solved are remaining and requiring improvement, and these compounds are not practically usable.

### SUMMARY OF THE INVENTION

As a result of intensive studies, the present inventors have found for the first time that out of the p-terphenyl compounds, only the compounds of the present invention not disclosed in related arts and having a very limited structure exert remarkably high hole transport ability and are useful for an electrophotographic photoreceptor. Furthermore, when the compound of the present invention is incorporated particularly into an electrophotographic photoreceptor, electrophotographic apparatus or process cartridge, sufficiently high sensitivity and high power as a next-generation photoreceptor can be achieved. In this way, the present invention has been accomplished.

That is, the above-described object of the present invention can be attained by the following methods.

<1> An arylamine compound represented by formula (1):

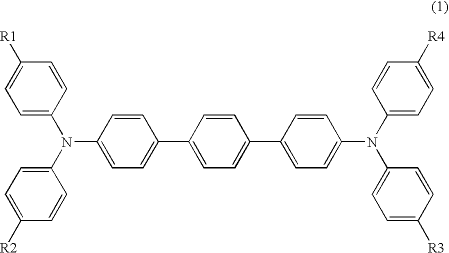

wherein R1 to R4 each independently represents a linear alkyl group or an arylalkenyl group, provided that when all of R1 to R4 represent linear alkyl groups, at least one of R1 to R4 represents a linear alkyl group having a carbon number of 3 or more.

<2> The arylamine compound as described in <1> above,

wherein R1 to R4 each independently represents a linear alkyl group having a carbon number of 1 to 20 or an alkenyl group having a carbon number of 2 to 20, which has an aryl group having a carbon number of 6 to 10 as a substituent, provided that when all of R1 to R4 represent linear alkyl groups each having a carbon number of 1 to 20, at least one of R1 to R4 represents a linear alkyl group having a carbon number of 3 or more.

<3> The arylamine compound as described in <2> above,

wherein R1 to R4 each independently represents a linear alkyl group having a carbon number of 1 to 8 or an arylalkenyl group with an alkenyl chain having a carbon number of 2 to 6, provided that when all of R1 to R4 represent linear alkyl groups each having a carbon number of 1 to 8, at least one of R1 to R4 represents a linear alkyl group having a carbon number of 3 or more.

<4> The arylamine compound as described in <3> above,

wherein R1 to R4 each independently represents a linear alkyl group having a carbon number of 1 to 6 or a mono- or di-arylalkenyl group with an alkenyl chain having a carbon number of 2 to 6, provided that when all of R1 to R4 represent linear alkyl groups each having a carbon number of 1 to 6, at least one of R1 to R4 represents a linear alkyl group having a carbon number of 3 or more.

<5> The arylamine compound as described in <4> above,

wherein R1 to R4 each independently represents a linear alkyl group having a carbon number of 1 to 4, and at least one of R1 to R4 represents a linear alkyl group having a carbon number of 3 or more.

<6> An electrophotographic photoreceptor, comprising:

an electrically conductive support; and

a photosensitive layer that comprises:
a charge generating material comprising at least one selected from the group consisting of selenium, selenium-tellurium, amorphous silicon, pyrylium salt-based dyes, azulenium-based dyes, cyanine-based dyes, squalium salt-based pigments, phthalocyanine-based pigments, anthanthrone-based pigments, dibenzopyrenequinone-based pigments, pyranthrone-based pigments, indigo-based pigments, quinacridone-based pigments, azo pigments and pyrrolopyrrole-based pigments; and
a charge transport material comprising at least one arylamine compound represented by formula (2):

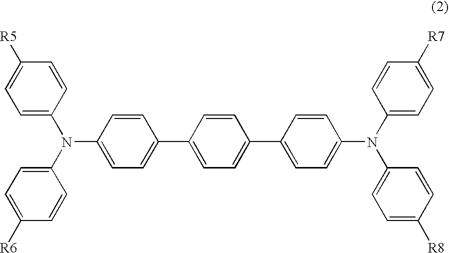

wherein R5 to R8 each independently represents an alkyl group, an arylalkyl group, an aryl group or an arylalkenyl group, provided that R5 to R8 do not represent aryl groups at the same time, and

when all of R5 to R8 represent alkyl groups, or at least one of R5 to R8 represents an alkyl group and the others of R5 to R8 represent aryl groups, at least one of R5 to R8 represents an alkyl group having a carbon number of 3 or more.

<7> The electrophotographic photoreceptor as described in <6> above,

wherein R5 to R8 each independently represents a linear alkyl group having a carbon number of 1 to 6, and at least one of R5 to R8 represents a linear alkyl group having a carbon number of 3 or more.

<8> The electrophotographic photoreceptor as described in <6> or <7> above,

wherein R5 and R7 are the same and R6 and R8 are the same, or R5 to R8 all are the same.

<9> The electrophotographic photoreceptor as described in <8> above,

wherein when R5 and R7 are the same and R6 and R8 are the same, a combination of groups represented by R5 and R7, and R6 and R8, is selected from the group consisting of methyl groups and n-propyl groups, methyl groups and n-butyl groups, ethyl groups and n-butyl groups, n-butyl groups and styryl groups, and n-pentyl groups and phenyl groups.

<10> The electrophotographic photoreceptor as described in any of <6> to <9> above,

wherein the photosensitive layer is a multilayer photosensitive layer that comprises;
a charge generating layer that comprises the charge generating material and has a thickness of 0.01 to 10 μm; and
a charge transport layer that comprises the charge transport material comprising the at least one arylamine compound represented by formula (2) and has a thickness of 1 to 100 μm.

### BRIEF DESCRIPTION OF THE DRAWINGS

FIG. 1 represents a partial schematic view of a multilayer electrophotographic photoreceptor showing one exemplary example of the layer construction of a multilayer electrophotographic photoreceptor using the charge transport material of the present invention;

FIG. 2 represents a partial schematic view of a multilayer electrophotographic photoreceptor showing another exemplary example of the layer construction of a multilayer electrophotographic photoreceptor using the charge transport material of the present invention;

FIG. 3 represents a partial schematic view of a multilayer electrophotographic photoreceptor showing one exemplary example of the layer construction of the multilayer electrophotographic photoreceptor of the present invention where an intermediate layer is provided;

FIG. 4 represents a partial schematic view of a multilayer electrophotographic photoreceptor showing another exemplary example of the layer construction of the multilayer electrophotographic photoreceptor of the present invention where an intermediate layer is provided;

FIG. 5 represents a partial schematic view of a multilayer electrophotographic photoreceptor showing one exemplary example of the layer construction of the multilayer electrophotographic photoreceptor of the present invention where an intermediate layer and an uppermost layer are provided;

FIG. 6 represents a partial schematic view of a single-layer electrophotographic photoreceptor showing one exemplary example of the layer construction of a single-layer electrophotographic photoreceptor using the charge transport material of the present invention; and

FIG. 7 represents a partial schematic view of a single-layer electrophotographic photoreceptor showing another exemplary example of the layer construction of a single-layer electrophotographic photoreceptor using the charge transport material of the present invention.

### DETAILED DESCRIPTION OF THE INVENTION

The present invention is described in detail below. The compound of the present invention is an arylamine compound represented by the following formula (1):

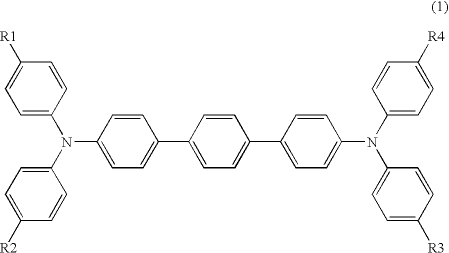

wherein R1 to R4 each independently represents a linear alkyl group or an arylalkenyl group, provided that when all of R1 to R4 represent linear alkyl groups, at least one of R1 to R4 represents a linear alkyl group having a carbon number of 3 or more.

In formula (1), R1 to R4 each is preferably a linear alkyl group having a carbon number of 1 to 20, such as methyl, ethyl, propyl, butyl, pentyl, hexyl, octyl, decyl, dodecyl, hexadecyl and octadecyl; or an alkenyl group having a carbon number of 2 to 20, which has an aryl group having a carbon number of 6 to 10 as a substituent, such as styryl, diphenylvinyl, naphthylvinyl, phenylbutadienyl, diphenylbutadienyl, phenylhexatrienyl and naphthyloctatetraenyl.

Among these, a linear alkyl group having a carbon number of 1 to 8, and an arylalkenyl group with the alkenyl chain having a carbon number of 2 to 6 are preferred, a linear alkyl group having a carbon number of 1 to 6 and a mono- or di-arylalkenyl group with the alkenyl chain having a carbon number of 2 to 6 are more preferred, and a linear alkyl group having a carbon number of 1 to 4 is still more preferred.

The electrophotographic photoreceptor of the present invention comprises a compound represented by the following formula (2):

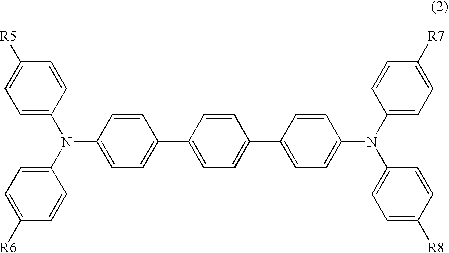

In formula (2), R5 to R8 each independently represents an alkyl group, an arylalkyl group, an aryl group or an arylalkenyl group, provided that R5 to R8 do not represent aryl groups at the same time, and

when all of R5 to R8 represent alkyl groups, or at least one of R5 to R8 represents an alkyl group and the others of R5 to R8 represent aryl groups, at least one of R5 to R8 represents an alkyl group having a carbon number of 3 or more.

In formula (2), R5 to R8 each is preferably a linear alkyl group having a carbon number of 1 to 20, such as methyl, ethyl, propyl, butyl, pentyl, hexyl, octyl, decyl, dodecyl, hexadecyl and octadecyl, a branched alkyl group having a carbon number of 1 to 20, such as isopropyl, isobutyl, sec-butyl, tert-butyl, neopentyl, isohexyl, tert-octyl, neodecyl, isotetradecyl and isooctadecyl; a cyclic alkyl group such as cyclopropyl, cyclobutyl, cyclopentyl, cyclohexyl and cycloheptyl; an alkyl group having a carbon number of 1 to 20, which has an aryl group having a carbon number of 6 to 10 as a substituent, such as benzyl, phenethyl, phenylpropyl, naphthylbutyl, phenanthrylpentyl, anthrylhexyl, phenyloctyl, diphenylmethyl, 3,3-diphenylpropyl, trityl and 4,4,4-triphenylbutyl; an aryl group having a carbon number of 6 to 14, such as phenyl, naphthyl, phenanthryl and anthryl; or an alkenyl group having a carbon number of 2 to 20, which has an aryl group having a carbon number of 6 to 10 as a substituent, such as styryl, diphenylvinyl, naphthylvinyl, phenylbutadienyl, diphenylbutadienyl, phenylhexatrienyl and naphthyloctatetraenyl. Among these, a linear alkyl group having a carbon number of 1 to 8, a monoarylalkyl group with the alkyl chain having a carbon number of 1 to 6, and an arylalkenyl group with the alkenyl chain having a carbon number of 2 to 6 are preferred, a linear alkyl group having a carbon number of 1 to 6 and a mono- or di-arylalkenyl group with the alkenyl chain having a carbon number of 1 to 6 are more preferred, and a linear alkyl group having a carbon number of 1 to 6 is still more preferred. By introducing a linear alkyl group into a substituent, pinholes and precipitations of crystals do not occur at the time of coating film formation, and then a good-quality organic thin film having high durability can be formed.

In general, when aryl groups are selected as substituents, the hole mobility improves, but solubility in an organic solvent and solubility with a binder resin decrease, thus it is not preferred. Because of this, in the present invention, all of R5 to R8 do not represent aryl groups at the same time. When groups represented by R5 to R8 consist of aryl groups and alkyl groups (i.e. at least one of R5 to R8 represents an alkyl group and the others of R5 to R8 represent aryl groups), at least one of R5 to R8 represents an alkyl group having a carbon number of 3 or more in order to prevent the decrease of solubility. Similarly, when all of R5 to R8 represent alkyl groups, because it adversely affects solubility, at least one of R5 to R8 represents an alkyl group having a carbon number of 3 or more.

The combination of R5 to R8 is preferably a combination where R5 and R7 are the same and R6 and R8 are the same or where R5 to R8 all are the same. When R5 and R7 are the same and R6 and R8 are the same, preferable combinations thereof include methyl groups and n-propyl groups, methyl groups and n-butyl groups, ethyl groups and n-butyl groups, n-butyl groups and styryl groups, and n-pentyl groups and phenyl groups.

Specific examples of the compound represented by formula (2) are shown in Table 1 below, but these are merely illustrative examples of the compound of the present invention, and the present invention is not limited to the compounds shown in Tables 1 to 3 below.

In Table 1, Me denotes a methyl group, Et denotes an ethyl group, n-Pr denotes an n-propyl group, n-Bu denotes an n-butyl group, sec-Bu denotes a sec-butyl group, tert-Bu denotes a tert-butyl group, and Ph denotes a phenyl group.

Also, at the synthesis of each compound, mass analysis (MS) was performed by an atmospheric pressure chemical ionization method. The protonated molecular ion peak ([M+H]⁺) obtained is shown in Table 1 below.

TABLE 1

| Compound No. | R5 | R6 | R7 | R8 | MS [M + H]⁺ |
|---|---|---|---|---|---|
| 1-1 | Me | n-Pr | Me | n-Pr | m/z676 |
| 1-2 | Me | n-Bu | Me | n-Bu | m/z704 |
| 1-3 | benzyl | n-pentyl | benzyl | n-pentyl | m/z884 |
| 1-4 | Me | n-hexyl | Me | n-hexyl | m/z760 |
| 1-5 | Me | cyclopentyl | Me | cyclopentyl | m/z728 |
| 1-6 | Me | 4-phenyl-1,3- | Me | 4-phenyl- | m/z848 |
| | | butadienyl | | 1,3- | |
| | | | | butadienyl | |
| 1-7 | Et | n-Pr | Et | n-Pr | m/z704 |
| 1-8 | Et | n-Bu | Et | n-Bu | m/z732 |
| 1-9 | Et | n-pentyl | Et | n-pentyl | m/z760 |
| 1-10 | Et | n-hexyl | Et | n-hexyl | m/z788 |
| 1-11 | Et | cyclopentyl | Et | cyclopentyl | m/z756 |
| 1-12 | Et | cyclohexyl | Et | cyclohexyl | m/z784 |
| 1-13 | n-Pr | isopropyl | n-Pr | isopropyl | m/z732 |
| 1-14 | isopropyl | tert-Bu | isopropyl | tert-Bu | m/z760 |
| 1-15 | n-Pr | n-hexyl | n-Pr | n-hexyl | m/z816 |
| 1-16 | isopropyl | n-decyl | isopropyl | n-decyl | m/z928 |
| 1-17 | n-Pr | cyclobutyl | n-Pr | cyclobutyl | m/z756 |
| 1-18 | n-Pr | cyclohexyl | n-Pr | cyclohexyl | m/z812 |
| 1-19 | n-Pr | Et | n-Pr | Ph | m/z752 |
| 1-20 | n-Bu | styryl | n-Bu | styryl | m/z880 |
| 1-21 | n-Bu | 4-phenyl-1,3- | n-Bu | 4-phenyl- | m/z932 |
| | | butadienyl | | 1,3- | |
| | | | | butadienyl | |
| 1-22 | n-Bu | n-tetradecyl | n-Bu | n-tetradecyl | m/z1068 |
| 1-23 | n-Bu | cyclopentyl | n-Bu | cyclopentyl | m/z812 |
| 1-24 | n-Bu | cyclohexyl | n-Bu | cyclohexyl | m/z840 |
| 1-25 | n-pentyl | Ph | n-pentyl | Ph | m/z856 |
| 1-26 | neopentyl | sec-Bu | neopentyl | sec-Bu | m/z816 |
| 1-27 | n-pentyl | n-hexyl | n-pentyl | n-hexyl | m/z872 |
| 1-28 | n-pentyl | naphthyl | n-pentyl | naphthyl | m/z956 |
| 1-29 | n-pentyl | cyclohexyl | n-pentyl | cyclohexyl | m/z868 |
| 1-30 | isopentyl | Ph | isopentyl | Ph | m/z856 |
| 1-31 | n-hexyl | n-hexyl | n-hexyl | n-hexyl | m/z900 |
| 1-32 | n-hexyl | cyclopentyl | n-hexyl | cyclopentyl | m/z868 |
| 1-33 | n-hexyl | cyclohexyl | n-hexyl | cyclohexyl | m/z896 |
| 1-34 | n-hexyl | 2,2- | n-hexyl | 2,2- | m/z1088 |
| | | diphenyl- | | diphenyl- | |
| | | vinyl | | vinyl | |
| 1-35 | cyclopentyl | cyclopentyl | cyclopentyl | cyclopentyl | m/z836 |
| 1-36 | cyclopentyl | 4,4- | cyclopentyl | 4,4- | m/z1108 |
| | | diphenyl- | | diphenyl- | |
| | | 1,3- | | 1,3- | |
| | | butadienyl | | butadienyl | |
| 1-37 | cyclopentyl | Ph | cyclopentyl | Ph | m/z852 |
| 1-38 | cyclohexyl | n-octyl | cyclohexyl | n-octyl | m/z952 |
| 1-39 | cyclohexyl | Ph | cyclohexyl | Ph | m/z880 |
| 1-40 | Me | Et | n-Pr | n-Bu | m/z704 |
| 1-41 | Me | Me | benzyl | benzyl | m/z772 |
| 1-42 | Me | Me | phenethyl | phenethyl | m/z800 |
| 1-43 | Me | Me | n-pentyl | n-pentyl | m/z676 |
| 1-44 | Me | Me | n-hexyl | n-hexyl | m/z760 |
| 1-45 | Me | Ph | cyclopentyl | Ph | m/z798 |
| 1-46 | Me | Me | cyclohexyl | cyclohexyl | m/z756 |
| 1-47 | Me | Me | 2,2- | 2,2- | m/z948 |
| | | | diphenyl- | diphenyl- | |
| | | | vinyl | vinyl | |
| 1-48 | phenethyl | phenethyl | phenethyl | phenethyl | m/z980 |
| 1-49 | Et | Et | n-Bu | n-Bu | m/z732 |
| 1-50 | Et | Et | n-pentyl | n-pentyl | m/z760 |
| 1-51 | Et | Et | n-hexyl | n-hexyl | m/z788 |
| 1-52 | Et | Et | cyclopentyl | cyclopentyl | m/z756 |
| 1-53 | Et | Et | cyclohexyl | cyclohexyl | m/z784 |
| 1-54 | Et | Et | 4-phenyl- | 4-phenyl- | m/z876 |
| | | | 1,3- | 1,3- | |
| | | | butadienyl | butadienyl | |
| 1-55 | isopropyl | isopropyl | n-Bu | n-Bu | m/z760 |
| 1-56 | n-Pr | n-Pr | n-pentyl | n-pentyl | m/z788 |
| 1-57 | n-Pr | n-Pr | n-hexyl | n-hexyl | m/z816 |
| 1-58 | n-Pr | Ph | cyclopentyl | cyclopentyl | m/z818 |
| 1-59 | n-Pr | n-Pr | cyclohexyl | cyclohexyl | m/z812 |
| 1-60 | n-Pr | n-Pr | Ph | Ph | m/z800 |
| 1-61 | n-Bu | n-Bu | n-pentyl | n-pentyl | m/z816 |
| 1-62 | n-Bu | n-Bu | n-hexyl | n-hexyl | m/z844 |
| 1-63 | n-Bu | n-Bu | cyclopentyl | cyclopentyl | m/z812 |
| 1-64 | n-Bu | n-Bu | cyclohexyl | cyclohexyl | m/z840 |
| 1-65 | n-Bu | n-Bu | styryl | styryl | m/z880 |
| 1-66 | n-pentyl | n-pentyl | n-hexyl | n-hexyl | m/z872 |
| 1-67 | n-octyl | n-octyl | cyclopentyl | cyclopentyl | m/z924 |
| 1-68 | n-pentyl | n-pentyl | cyclohexyl | cyclohexyl | m/z868 |
| 1-69 | n-decyl | n-decyl | naphthyl | naphthyl | m/z1096 |
| 1-70 | n-hexyl | n-hexyl | cyclopentyl | cyclopentyl | m/z868 |
| 1-71 | n-hexyl | n-hexyl | cyclohexyl | cyclohexyl | m/z896 |
| 1-72 | n-hexyl | n-hexyl | 6,6- | 6,6- | m/z1192 |
| | | | diphenyl- | diphenyl- | |
| | | | 1,3,5- | 1,3,5- | |
| | | | hexatrienyl | hexatrienyl | |
| 1-73 | cyclopentyl | cyclopentyl | cyclohexyl | cyclohexyl | m/z864 |
| 1-74 | cyclopentyl | cyclopentyl | Ph | Ph | m/z852 |
| 1-75 | cyclohexyl | cyclohexyl | naphthyl | naphthyl | m/z980 |

The compounds (1) and (2) of the present invention can be synthesized by combining conventionally known methods. One example of the synthesis method is described below by referring to a synthesis method when in the compound (2), R5 and R7 are the same and R6 and R8 are the same.

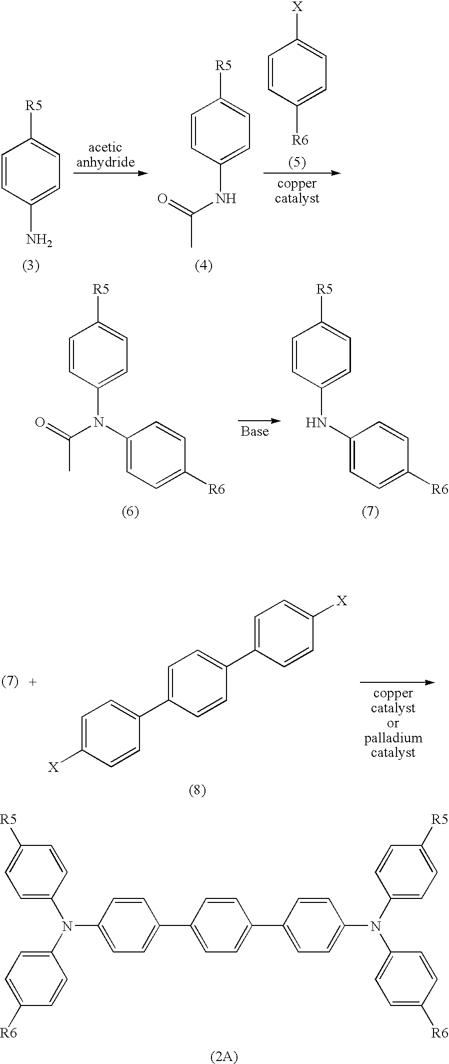

In these formulae, R5 and R6 have the same meanings as above and X represents a chlorine, bromine or iodine atom.

In the synthesis method above, an aniline compound (3) is reacted with acetic anhydride to synthesize an amide compound (4). The obtained amide compound (4) and a halogenobenzene compound (5) are caused to derive a compound (6) by an Ullmann reaction, and a diarylamine compound (7) is then obtained by the hydrolysis with a base. This compound is further reacted with a dihalo-p-terphenyl (8) in the presence of a copper catalyst (see, for example, JP-A-2005-350416) or subjected to a coupling reaction with a dihalo-p-terphenyl (8) by using a palladium catalyst (see, J. Org. Chem., Vol. 65, page 5327 (2000)), whereby the objective compound (2A) can be obtained.

In the above, the aniline compound (3) as a starting material is easily available from various commercial products. The dihalo-p-terphenyl (8) as another starting material is also easily derived from p-terphenyl by a known method (see, for example, JP-A-11-128739).

On the other hand, when the substituents R5 to R8 all are the same, aniline compounds (3) are mutually condensed in the presence of an aluminum chloride catalyst to obtain a diarylamine compound (7a), and this compound is reacted with a dihalo-p-terphenyl (8) by the above-described method, whereby the objective compound (2a) can more simply synthesized.

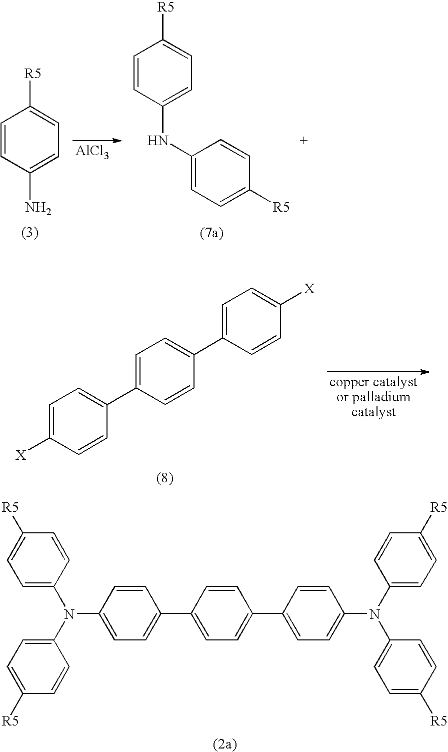

In these formulae, R5 and X have the same meanings as above.

The conditions in these two reactions, such as molar ratio of raw materials, reaction solvent and reaction temperature, may be selected in accordance with known methods.

Incidentally, even in the case where R5 to R8 all are different or in the case of a combination of R5 to R8 other than those described above, the objective compound can be synthesized according to the above-described synthesis methods.

The object of the present invention can be attained by incorporating the objective compound (2) into an electrophotographic photoreceptor. In the present invention, it is sufficient if at least one kind of the objective compound (2) is contained in the electrophotographic photoreceptor, and two or more kinds of the compound may be appropriately combined and used. Also, the compound may be used by appropriately combining it with a known charge transport material other than that of the present invention.

Specific examples of the compound which can be used as the charge transport material in the present invention include the compounds described in the following documents. Examples of the hole-transporting low molecular material include triphenylmethane derivatives described in JP-B-45-555 (the term “JP-B” as used herein means an “examined Japanese patent publication”) and JP-A-59-38752; hydrazone derivatives described in JP-A-54-150128, JP-A-54-150158, JP-A-55-42380, JP-A-55-46760, JP-A-57-11350, JP-A-57-67940, JP-A-57-101844, JP-A-58-159536, JP-A-58-184947, JP-A-58-159539, JP-A-59-15251, JP-A-60-146248, JP-A-60-19148, JP-A-61-23154, JP-A-63-151955, JP-A-1-102469, JP-B-2-47741, JP-A-2-210451, JP-A-3-91760, JP-A-3-278061, JP-A-4-246652, JP-A-5-188609 and JP-A-7-152187; styryl derivatives described in JP-A-57-148750, JP-A-58-65440, JP-A-58-198043, JP-A-58-198425, JP-A-59-216853, JP-A-60-196768, JP-A-61-14642, JP-A-62-30255, JP-A-62-287257, JP-A-63-225660, JP-A-63-316867, JP-A-4-290852, JP-A-6-273950, JP-A-7-173112, JP-A-8-295655, JP-A-2000-219657, JP-A-2000-219659, JP-A-2000-306671, JP-A-2000-327598, JP-A-2003-300941 and JP-A-2004-252001; triarylamine derivatives described in JP-B-58-32372, JP-A-61-124949, JP-A-1-118141, JP-A-1-142642, JP-A-1-280763, JP-A-2-36270, JP-A-2-230255, JP-A-5-119489, JP-A-5-150481, JP-A-6-329600, JP-A-8-20770, JP-A-8-20771, JP-A-8-179526, JP-A-2000-186066, JP-A-2000-309566, JP-A-2000-323281, JP-A-2000-327664, JP-A-2001-335542, JP-A-2001-335543, JP-A-2001-350282, JP-A-2002-20354, JP-A-2002-75655 and JP-A-2002-80433; and heterocyclic compounds described in JP-B-34-5466, JP-B-52-4188, JP-A-56-123544, JP-A-59-185341, JP-A-60-237454, JP-A-5-232721, JP-A-2000-268975, JP-A-2000-282025, JP-A-2001-89680, JP-A-2001-354668, JP-A-2002-173488 and JP-A-2003-13054.

Examples of the hole-transporting polymer material include polymer materials described in JP-B-34-10966, JP-A-61-170747, JP-A-64-13061, JP-A-1-1728, JP-A-1-9964, JP-A-1-13061, JP-A-1-19049, JP-A-1-241559, JP-A-2-151605, JP-A-4-11627, JP-A-4-175337, JP-A-4-183719, JP-A-4-225014, JP-A-4-230767, JP-A-4-320420, JP-A-5-232727, JP-A-5-310904, JP-A-6-234836, JP-A-6-234837, JP-A-6-234838, JP-A-6-234839, JP-A-6-234840, JP-A-6-234841, JP-A-6-239049, JP-A-6-236050, JP-A-6-236051, JP-A-6-295077, JP-A-7-56374, JP-A-8-176293, JP-A-8-208820, JP-A-8-211640, JP-A-8-227165, JP-A-8-253568, JP-A-8-269183, JP-A-9-62019, JP-A-9-43883, JP-A-9-71642, JP-A-9-87376, JP-A-9-104746, JP-A-9-110974, JP-A-9-110976, JP-A-9-157378, JP-A-9-221544, JP-A-9-227669, JP-A-9-235367, JP-A-9-241369, JP-A-9-268226, JP-A-9-272735, JP-A-9-302084, JP-A-9-302085, JP-A-9-328539, JP-A-2000-215986, JP-A-2002-56982, JP-A-2002-69161, JP-A-2002-75654, JP-A-2002-117982, JP-A-2002-117983, JP-A-2002-214389, JP-A-2002-214390, JP-A-2003-7470, JP-A-2003-17266, JP-A-2003-17270, JP-A-2003-36979, JP-A-2003-178884, JP-A-2003-208986 and JP-A-2003-257669.

Other specific examples of the known charge-transporting compound which can be used in combination with the compound of the present invention are shown below, but the charge-transporting compound usable in the present invention is not limited thereto.

### <Hole-Transporting Low Molecular Compound>

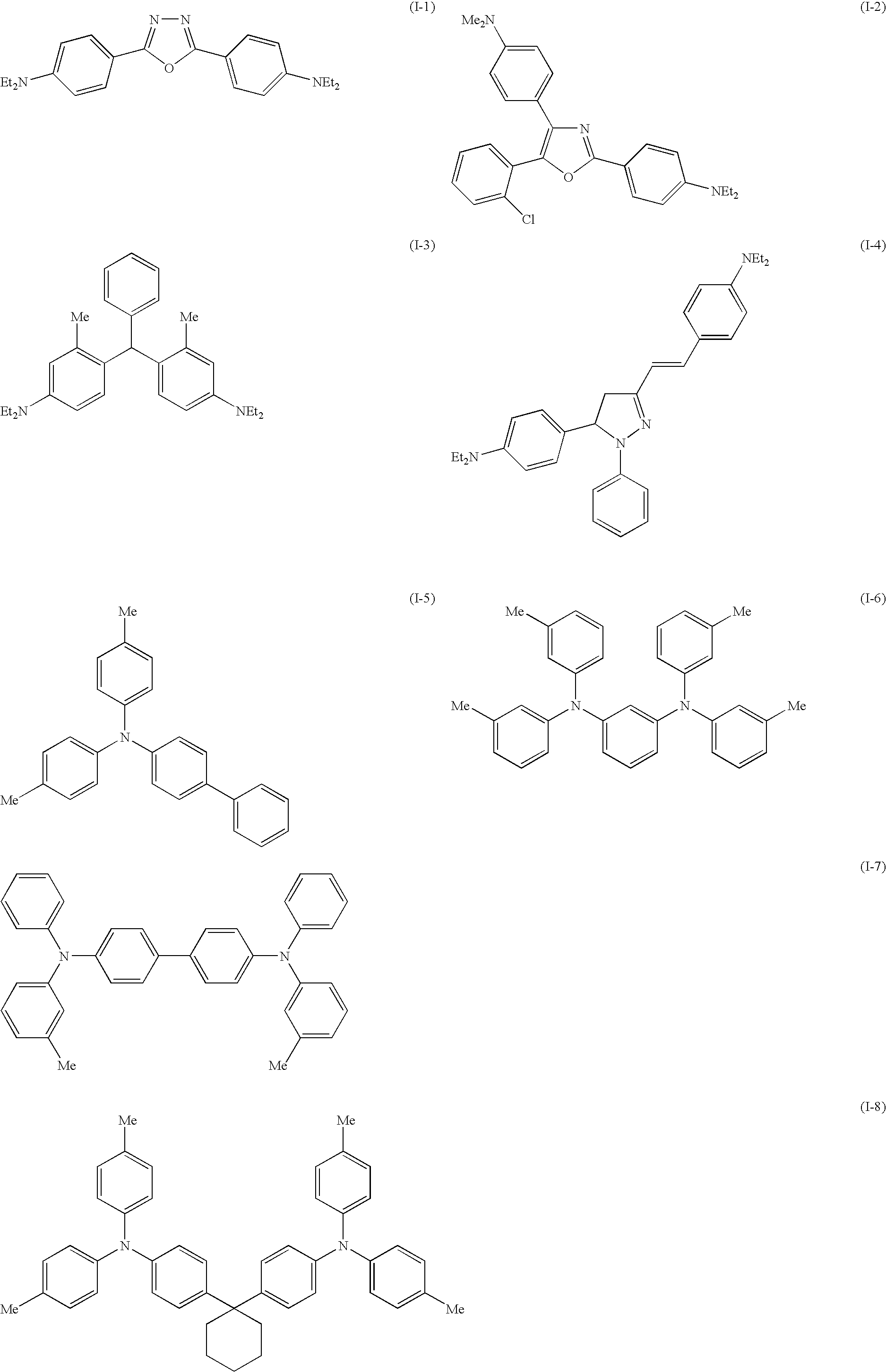

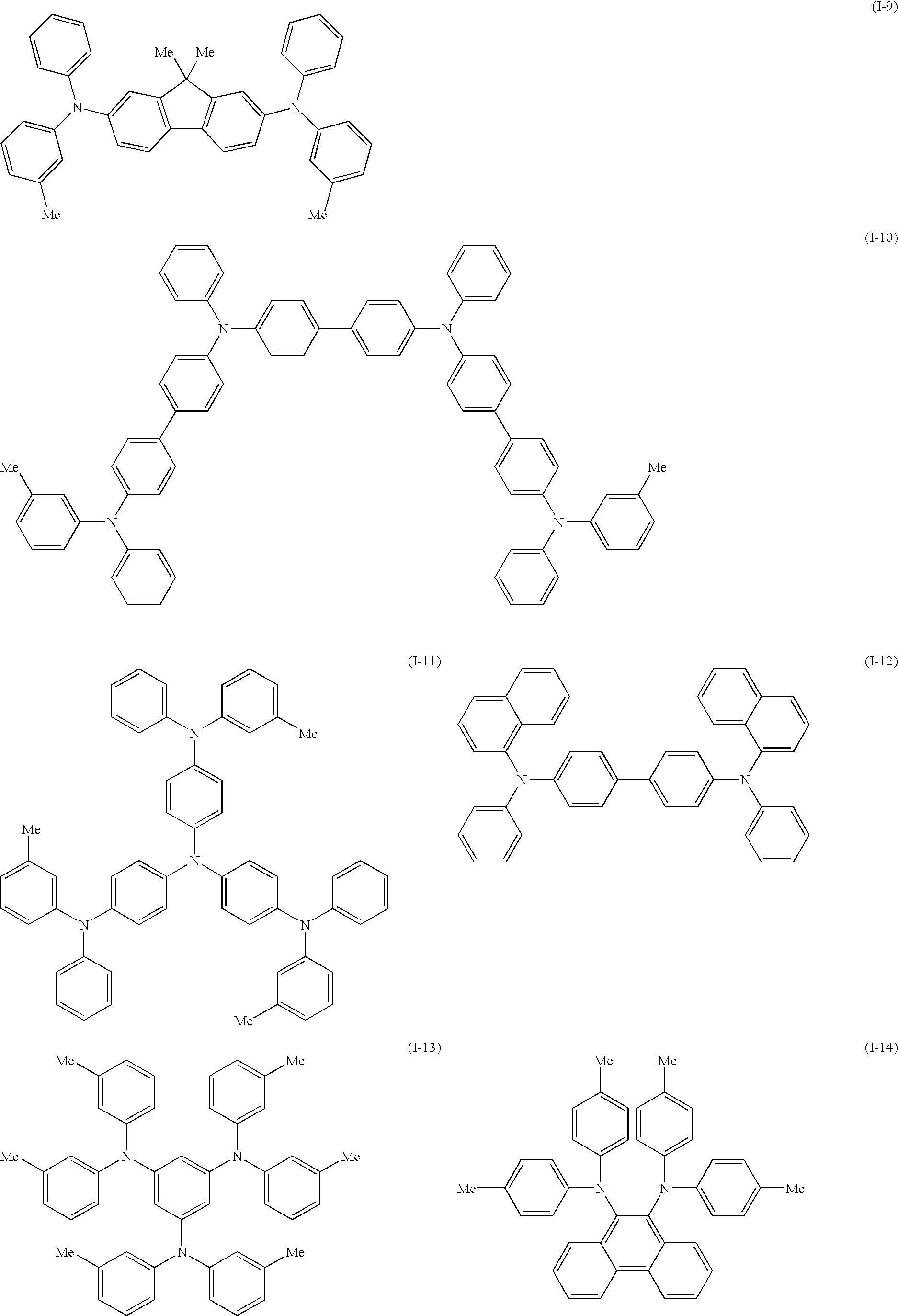

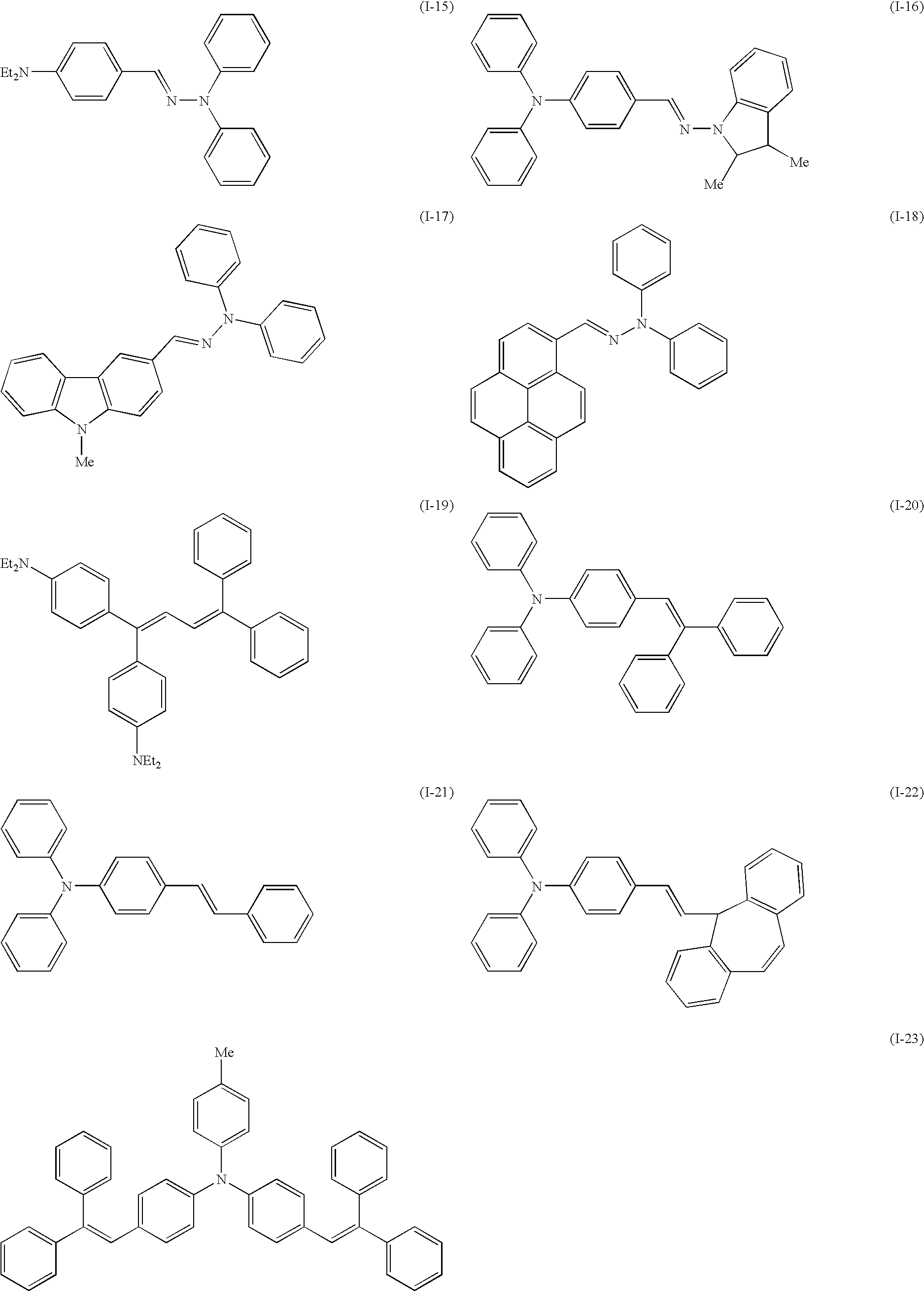

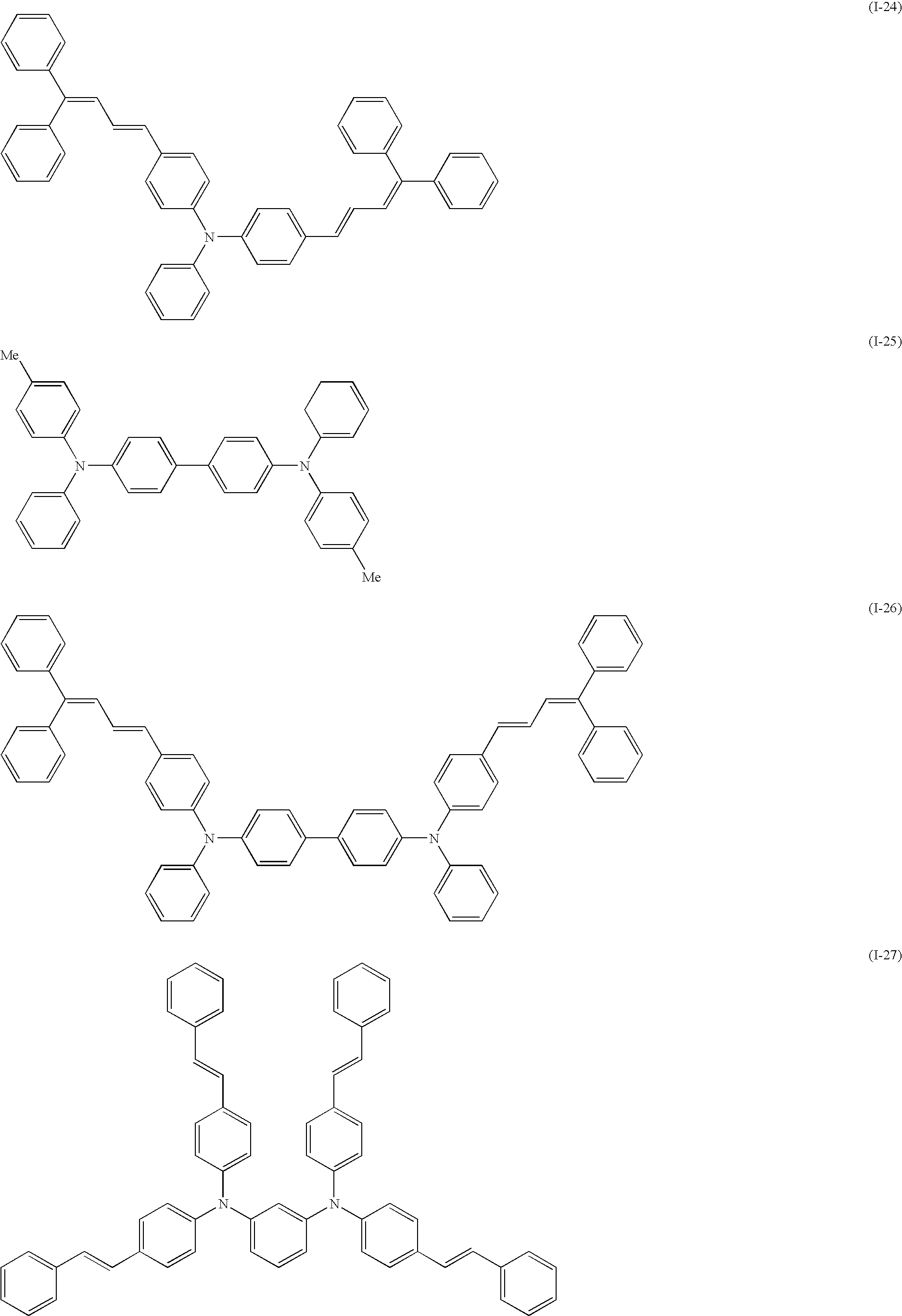

### <Hole-Transporting Polymer Compound>

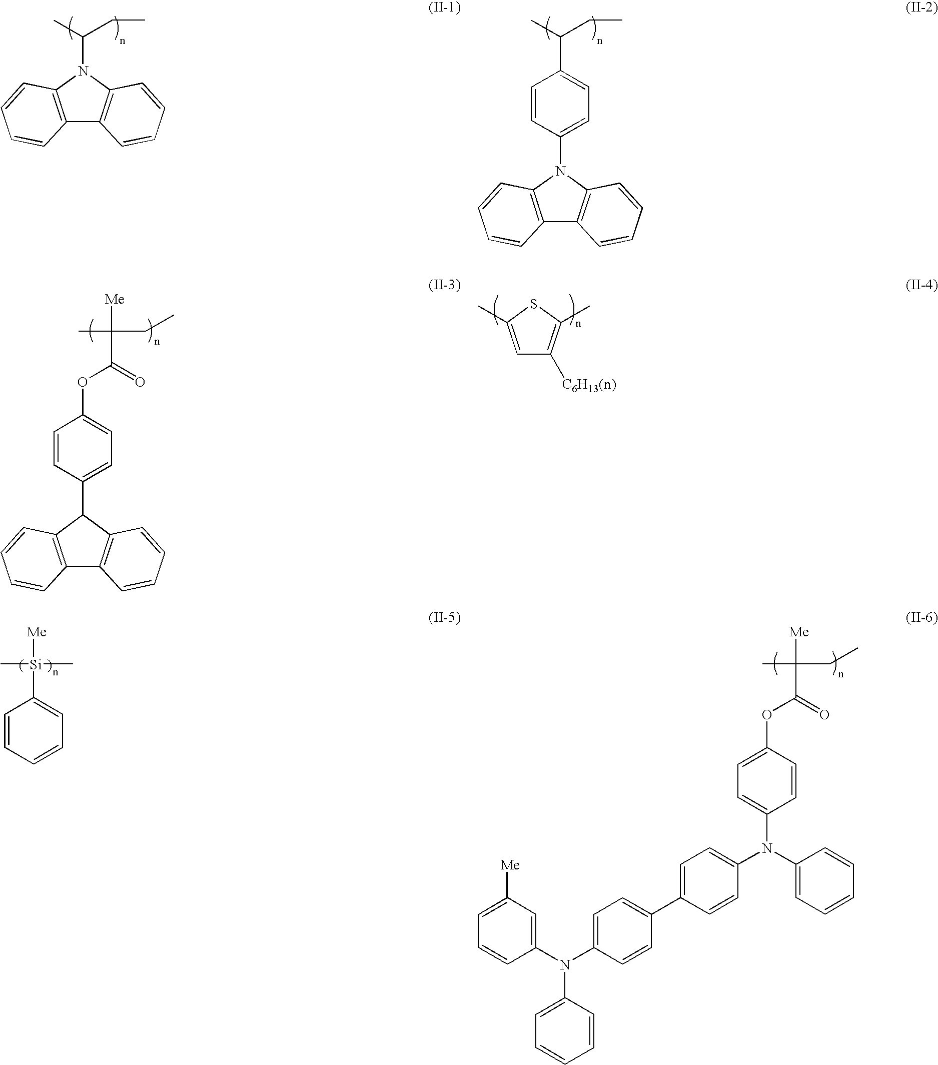

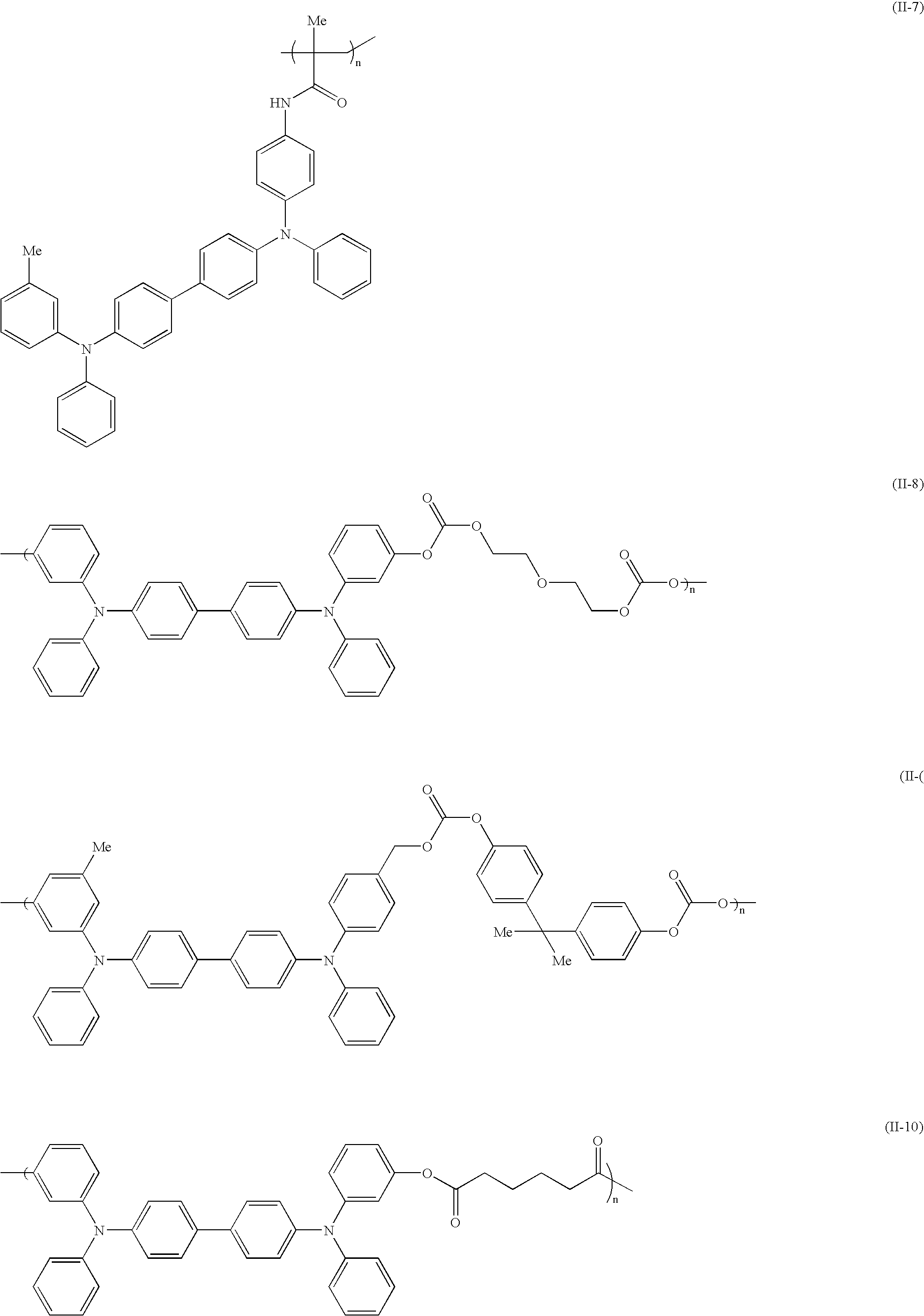

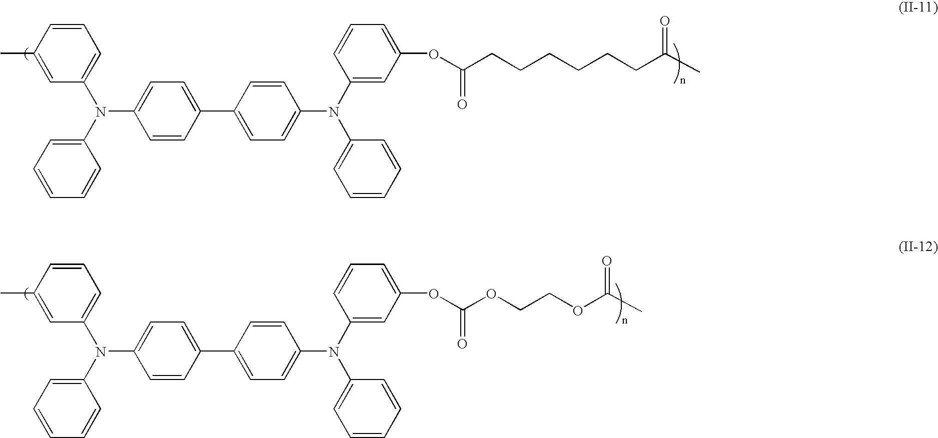

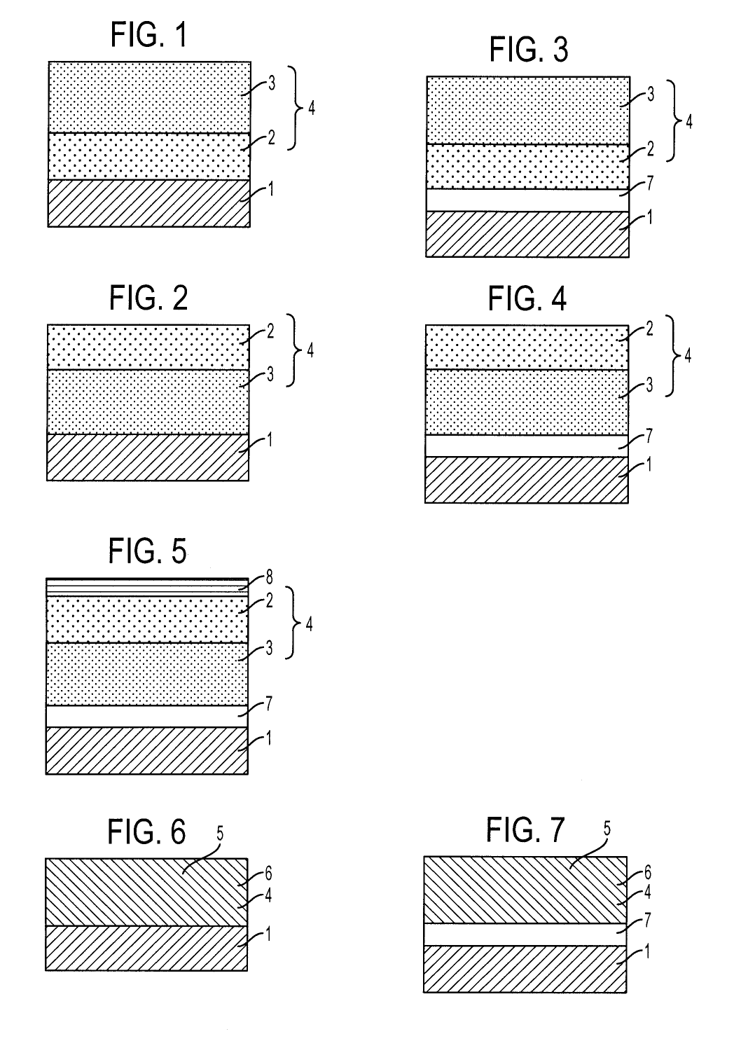

In the electrophotographic photoreceptor, at least one kind of the compound represented by formula (2) is contained as a charge transport agent and is used, if desired, in combination with an existing charge transport agent shown above. The mode of the electrophotographic photoreceptor of the present invention includes a multilayer photoreceptor where at least a charge generating layer and a charge transport layer are provided as the photosensitive layer on an electrically conductive support, and a single-layer photoreceptor where at least a layer containing a charge generating material and a charge transport material is provided as the photosensitive layer on an electrically conductive support. The constructions of these electrophotographic photoreceptors are described below. FIGS. 1 and 2 each is a schematic cross-sectional view showing the layer construction of a multilayer electrophotographic photoreceptor. The photosensitive layer 4 of the photoreceptor shown in FIG. 1 comprises a charge generating layer 2 provided on an electrically conductive support 1 and a charge transport layer 3 provided on the charge generating layer 2. The photosensitive layer 4 in FIG. 2 comprises a charge transport layer 3 provide on an electrically conducive support 1 and a charge generating layer 2 provided on the charge transport layer 3. In the multilayer electrophotographic photoreceptor, either a charge generating layer or a charge transport layer may be a layer closer to the support, but a layer construction of FIG. 1 where a charge transport layer is provided on a charge generating layer is preferred. FIG. 3 is a schematic cross-sectional view showing the layer construction of a multilayer electrophotographic photoreceptor where an undercoat layer 7 is provided between an electrically conductive support 1 and a charge generating layer 2. FIG. 4 is a schematic cross-sectional view showing the layer construction of a multilayer electrophotographic photoreceptor where an undercoat layer 7 is provided between an electrically conductive support 1 and a charge transport layer 3. FIG. 5 is a schematic cross-sectional view showing the layer construction of a multilayer electrophotographic photoreceptor where an undercoat layer 7 is provided between an electrically conductive support 1 and a charge transport layer 3 and an overcoat layer 8 is provided on a charge generating layer 2. FIGS. 6 and 7 each is a schematic cross-sectional view showing the layer construction of a single-layer electrophotographic photoreceptor. In FIG. 6, a photosensitive layer 4 containing a charge transport material 5 and a charge generating material 6 is provided on an electrically conductive support 1. In FIG. 7, an undercoat layer 7 is provided between an electrically conductive support 1 and a photosensitive layer 4. The electrophotographic photo-receptor of the present invention contains a compound of formula (2) in the photoreceptor, and the compound of formula (2) is preferably used as a charge transport material in the photosensitive layer provided on the electrically conductive support of the electrophotographic photoreceptor.

The above-described photoreceptor of the present invention can be produced by a commonly employed method. For example, in the case of a multilayer type, a compound of formula (2) is dissolved together with a binder resin in a solvent, and the resulting solution is coated on an electrically conductive support or a charge generating layer and dried to form a photosensitive layer. In this case, the film thickness of the charge generating layer is usually from 0.01 to 10 μm, preferably from 0.05 to 5 μm, more preferably from 0.1 to 2 μm. The film thickness of the charge transport layer is usually from 1 to 100 μm, preferably from 5 to 75 μm, more preferably from 10 to 40 μm. In another method, the photosensitive layer can be formed by vacuum-depositing a compound of formula (2) on an electrically conductive support or a charge generating layer. In the case of a single-layer type, a compound of formula (2) and other additives are dissolved together with a binder resin in a solvent, and the resulting solution is coated on an electrically conductive support and dried to form a photosensitive layer, whereby a single-layer electrophotographic photoreceptor can be produced. In the case of a single-layer type, the film thickness of the layer is usually from 1 to 100 μm, preferably from 5 to 75 μm. The compound of the present invention exerts a sufficiently high performance in both a multilayer type and a single-layer type but is preferably used in a multilayer type. In the photoreceptor produced as above, an undercoat layer, an intermediate layer, a protective layer and the like can be provided, if desired.

Incidentally, the compounds of the present invention can be used for various semiconductor devices in addition to an electrophotographic photoreceptor, and a device can be produced by incorporating at least one kind of the compound of the present invention into an organic thin-film layer of hole injection layer, hole transport layer or light-emitting layer and interposing the thin film between a pair of electrodes. The form of the organic semiconductor device includes, for example, an organic electroluminescent device having a device configuration such as anode/hole injection-transport layer/light-emitting layer/electron injection-transport layer/cathode, anode/hole injection-transport layer/light-emitting layer/cathode, anode/light-emitting layer/electron injection-transport layer/cathode, and anode/light-emitting layer/cathode; a photoelectric conversion device as represented by an organic image sensor having a device configuration such as anode/charge generating layer/hole transport layer/cathode, and a solar cell having a device configuration such as anode/electron transport layer/hole transport layer/cathode; and an organic transistor device having a device configuration such as gate electrode/insulating layer/source•drain electrode/hole transport layer.

In the case of using the compound of formula (2) for usage other than an electrophotographic photoreceptor, for example, in an organic electroluminescent element, the film thickness of each of the hole injection/transport layer, light-emitting layer and electron injection/transport layer is usually from 0.001 to 3 μm, preferably from 0.005 to 1 μm, more preferably from 0.01 to 0.5 μm. In the case of an organic image sensor, the film thickness of each of the charge generating layer and hole transport layer is usually from 0.001 to 5 μm, preferably from 0.01 to 3 μm, more preferably from 0.1 to 1 μm. In the case of a solar cell, the film thickness of each of the electron transport layer and hole transport layer is usually from 0.001 to 0.5 μm, preferably from 0.01 to 0.3 μm, more preferably from 0.03 to 0.1 μm. In the case of an organic transistor device, the film thickness of the hole transport layer is usually from 0.001 to 5 μm, preferably from 0.01 to 3 μm, more preferably from 0.1 to 1 μm.

Examples of the binder resin for forming a charge transport layer in the electrophotographic photoreceptor include various resins such as polycarbonate resin, polyarylate resin, polyester resin, methacrylic resin, acrylic resin, polyvinyl chloride resin, polyvinylidene chloride resin, polystyrene resin, polyvinyl acetate resin, styrene-butadiene copolymer, vinylidene chloride-acrylonitrile copolymer, vinyl chloride-vinyl acetate copolymer, vinyl chloride-vinyl acetate-maleic anhydride copolymer, silicone resin, silicone-alkyd resin, phenol-formaldehyde resin, styrene-alkyd resin, poly-N-carbazole, polyvinylbutyral, polyvinylformal, polysulfone, casein, gelatin, polyvinyl alcohol, phenol resin, polyamide, polyphenylene oxide resin, polyurethane resin, cellulose ester resin, phenoxy resin and epoxy resin. These binder resins each may be used alone, or two or more thereof may be used in appropriate combination. Among these binder resins, a polycarbonate resin and a polyarylate resin are preferred because of their excellence in terms of compatibility with a charge transport material, solubility in a solvent, and mechanical strength. The amount of the binder resin used is from 0.1 to 10 times, preferably from 0.3 to 5.0 times, the mass of the compound of formula (2).

The solvent for dissolving the compound of formula (2) and the binder resin is not particularly limited, but examples thereof include a polar organic solvent such as tetrahydrofuran, 1,4-dioxane, methyl ethyl ketone, cyclohexanone, acetonitrile, N,N-dimethylformamide and ethyl acetate; an aromatic organic solvent such as toluene and xylene; and a halogenated hydrocarbon solvent such as dichloromethane, dichloroethane, chloroform, carbon tetrachloride, chlorobenzene and dichlorobenzene. These solvents each may be used alone, or two or more thereof may be used in appropriate combination.

In coating the compound of formula (2) on an electrically conductive support or a charge generating layer, for example, a compound of formula (2), optionally other charge transport materials and, if desired, further additives such as plasticizer, surface lubricant, potential stabilizer, antioxidant, ultraviolet absorbent and sensitizer, are dissolved or dispersed in a solvent to prepare a coating solution, and this coating solution is coated using a known coating method. As regards the method for dispersing the components, a general dispersion method such as ball mill, sand mill, colloid mill, Dyno-mill, jet mill, attritor, vibration mill and ultrasonic disperser is used. Also, examples of the coating method include a dip coating method, a spray coating method, a spinner coating method, a wire bar coating method, a blade coating method, a roller coating method, a bead coating method, an air knife coating method and a curtain coating method. After the coating, the film coating is dried at room temperature and further dried under heating usually at 30 to 200° C. to form a charge transport layer.

In the electrophotographic photoreceptor of the present invention, in the case of a single-layer type, a charge transport material including a compound of formula (2) and a charge generating material are contained in the photosensitive layer, and in the case of a multilayer type, a charge transport layer and a charge generating layer are provided. As for the charge generating material, those conventionally used for electrophotographic photoreceptors can be employed. Examples thereof include an inorganic charge generating material such as selenium, selenium-tellurium and amorphous silicon; and an organic charge generating material such as cation dye (e.g., pyrylium salt-based dye, thiapyrylium salt-based dye, azulenium based dye, thiacyanine-based dye, quinocyanine-based dye), polycyclic quinone pigment (e.g., squalium salt-based pigment, phthalocyanine-based pigment, anthanthrone-based pigment, dibenzopyrenequinone-based pigment, pyranthrone-based pigment), indigo-based pigment, quinacridone-based pigment, azo pigment and pyrrolopyrrole-based pigment. These charge generating materials each may be used alone or two or more thereof may be used in appropriate combination. Among these, preferred are a phthalocyanine pigment such as alkoxytitanium phthalocyanine, oxotitanium phthalocyanine, copper phthalocyanine, nonmetallic phthalocyanine, chlorogallium phthalocyanine, chloroindium phthalocyanine, hydroxygallium phthalocyanine and vanadyl phthalocyanine; and an azo pigment such as monoazo pigment, bisazo pigment and trisazo pigment.

In the case of forming the charge generating material by coating, similarly to the formation of charge transport layer, a chare generating material together with a binder polymer and, if desired, various additives are dissolved or dispersed in a solvent to prepare a coating solution, and this coating solution is coated on an electrically conductive support or a charge transport layer and dried to form a photosensitive layer of 0.01 to 10 μm in thickness. The film thickness of the photosensitive layer is preferably from 0.05 to 5 μm, more preferably from 0.1 to 2 μm. The binder polymer and additives used here may be the same as those used for the formation of charge transport layer, and the formation method of layer and the formation conditions are also the same as those in forming the charge transport layer.

The electrically conductive support for use in the electrophotographic photoreceptor of the present invention is not particularly limited, and examples thereof include a metallic drum such as aluminum, copper, iron, zinc and nickel; a drum-like, sheet-like or plate-like support treated to be electrically conductive by vapor-depositing a metal such as aluminum, copper, gold, silver, platinum, palladium, titanium, nickel, nickel-chromium, stainless steel and copper-indium, on polymer-made sheet, paper, plastic or glass; a drum-like, sheet-like or plate-like support treated to be electrically conductive by vapor-depositing an electrically conductive metal compound such as indium tin oxide or laminating a metal foil on a substrate such as polymer-made sheet, paper, plastic or glass; and a drum-like, sheet-like or plate-like support treated to be electrically conductive by dispersing carbon black, indium oxide, tin oxide-antimony oxide powder, or copper iodide in a binder resin and coating it on polymer-made sheet, paper, plastic or glass. The support may be formed in an appropriate thickness, but the thickness is usually from 50 to 1,000 μm.

The electrophotographic photoreceptor of the present invention is a photoreceptor having high charge mobility and having excellent electrophotographic properties such as high sensitivity, high durability and high power, and is suitably used in various electrophotographic fields such as analogue copying machine, digital copying machine (e.g., black-and-white, multicolor, full color), various printers (e.g., laser, LED, liquid crystal shutter), platemaker and facsimile.

An image forming device using the electrophotographic photoreceptor of the present invention is described below.

The electrophotographic photoreceptor of the present invention can be fabricated as an electrophotographic apparatus equipped with at least one member out of a charging device, a developing device, a transfer device and, if desired, a cleaning device.

The image forming process of the electrophotographic system is roughly classified into the followings.

A first step is a charging process, and this is a step of applying an electric charge to the surface of an electrophotographic photoreceptor, thereby uniformly charging the surface to a fixed potential. The charging method includes non-contact charging as represented by corona charging such as corotron and scorotron, and contact charging as represented by electrically conductive brush charging and electrically conductive roller charging. In the present invention, both charging methods can be used.

A second step is an exposure process, where an original is irradiated with various gas lasers, a fluorescent tube, a halogen lamp, a fluorescent lamp, LED, a semiconductor laser or the like and the photoreceptor surface is exposed to the reflected light by using an optical system to form an electrostatic latent image. In the present invention, all of those exposure means can be employed.

A third step is a developing process, and this is a step of electrostatically attaching toner to the electrostatic latent image formed on the photoreceptor surface to give a visible image. The development system includes a dry development system such as cascade development, one-component insulating toner development, one-component electrically conductive toner development and two-component magnetic brush development, and a wet development system using liquid toner or the like. In the present invention, both systems can be employed.

A fourth step is a transfer process and in this process, a toner image formed on the photoreceptor surface is transferred and attached to recording paper. Examples of the method therefor include an electrostatic transfer method such as corona transfer, roller transfer and belt transfer, a pressure transfer method, and an adhesive transfer method. In the present invention, all of these methods can be used.

Also, toner is remaining on the photoreceptor surface after the transfer step, and a cleaning process of removing the remaining toner or other adhering matters is necessary. Therefore, in an electrophotographic apparatus or a process cartridge-type electrophotographic apparatus, a cleaning device is usually equipped together with the electrophotographic photoreceptor. Examples of the cleaning method include a brush cleaner, a magnetic brush cleaner, a magnetic roller cleaner and a blade cleaner. In the present invention, all of these methods can be used.

As for various devices in the image forming apparatus, specifically, a charging device for charging the electrophotographic photoreceptor, an exposure device for exposing the charged electrophotographic photoreceptor, a developing device for developing the electrostatic latent image to form a toner image, a transfer device for transferring the toner image to a transfer medium from the electrophotographic photoreceptor, and a cleaning device for removing toner adhering to the electrophotographic photoreceptor after transfer, there can be used generally employed devices (see, for example, Denshi-Shashin Gijutsu no Kiso to Oyo (Basics and Applications of Electrophotographic Technology), compiled by The Society of Electrophotography of Japan, 1st ed., Corona Publishing Co., Ltd. (1988)).

Incidentally, the image forming apparatus of the present invention is not limited to this construction and, for example, in addition to the construction above, a fixing device by an arbitrary system such as heated roller fixing, flash fixing, oven fixing and pressure fixing, and a destaticizing device of removing the charge by exposing the electrophotographic photoreceptor with use of a fluorescent tube, LED or the like may be added.

Also, the above-described image forming apparatus may be modified into a construction for offset printing or a construction for a full color tandem system using a plurality of kinds of toners.

Furthermore, the electrophotographic photoreceptor of the present invention may be fabricated as a process cartridge equipped with at least one member out of a charging device, a developing device, a transfer device and a cleaning device.

### EXAMPLES

The present invention is described in greater detail below by referring to Examples, but the present invention is not limited to the following Examples. The objective products were each identified by the at least one measurement of IR, NMR and above-mentioned MS.

### Synthesis Example 1

### Synthesis of Compound No. 1-2

Toluene (70 ml) and 78.7 g (0.53 mol) of 4-n-butylaniline were charged, 55.5 g (0.54 mol) of acetic anhydride was added dropwise thereto under cooling, and thereafter, reaction was allowed to proceed for 1 hour at an inner temperature of 90 to 95° C. The reaction solution was cooled, 160 ml of water was added thereto, and the resulting solution was stirred, left standing and then subjected to liquid separation into an organic layer and an aqueous layer. The organic layer was removed by distillation under reduced pressure, the residue was crystallized by adding 200 ml of ethanol and 200 ml of water thereto and through separation by filtration, 91.1 g (yield: 90.3%) of N-acetyl-4-n-butylaniline was obtained as a white crystal.

Subsequently, 83.7 g (0.44 mol) of N-acetyl-4-n-butylaniline, 121.4 g (0.71 mol) of 4-bromotoluene, 1.09 g (0.004 mol) of copper sulfate pentahydrate and 35.3 g (0.33 mol) of sodium carbonate were charged, the temperature thereof was elevated to 210 to 220° C., and reaction was allowed to proceed for 8 hours in a nitrogen atmosphere while further adding dropwise 255.1 g (1.49 mol) of 4 bromotoluene. After the reaction, 92 ml of water was added, and the resulting solution was stirred, left standing and then subjected to liquid separation into an organic layer and an aqueous layer. Thereafter, 40 ml of ethanol and 51.6 g (0.92 mol) of potassium hydroxide were added to the organic layer, and reaction was further allowed to proceed for 1 hour at 90 to 95° C. Furthermore, 76 ml of water and 100 ml of hexane were added thereto, and the resulting solution was stirred, left standing and then subjected to liquid separation into an organic layer and an aqueous layer. The organic layer was removed by distillation under reduced pressure, and 70.0 g (yield: 79.6%) of N-(n-butylphenyl)-N-tolyl-4-amine was obtained by distillation under reduced pressure as a fraction at a pressure reduction degree of 1 to 2 Torr and a tower temperature of 166 to 170° C.

Subsequently, 14.8 g (0.03 mol) of diiodo-p-terphenyl, 21.5 g (0.09 mol) of N-(n-butylphenyl)-N-tolyl-4-amine, 0.059 g (0.24 mmol) of copper sulfate pentahydrate, 8.4 g (0.06 mol) of potassium carbonate and 0.042 g (0.24 mmol) of L-ascorbic acid were charged and allowed to react for 5 hours at an inner temperature of 220 to 230° C. in a nitrogen atmosphere. After adding 80 ml of toluene and 40 ml of water thereto, the reaction solution was stirred, left standing and subjected to liquid separation into an organic layer and an aqueous layer. The organic layer was removed by distillation under reduced pressure, the residue was crystallized by adding 90 ml of ethyl acetate and 40 ml of methanol thereto and through separation by filtration, a slightly yellow crude crystal was obtained. The crude crystal was recrystallized from ethyl acetate to obtain 16.7 g (yield: 76.8%) of the objective compound.

### Example 1

### Measurement of Mobility

Amorphous selenium was vacuum-deposited on an aluminum substrate to a thickness of 0.5 μm, a solution obtained by dissolving a mixture of Compound (1-2) and Polycarbonate A in dichloromethane was coated thereon, and the coating was dried under pressure at 40° C. for 2 hours to form a photosensitive layer of 10 μm in thickness. A gold electrode was vapor-deposited on the surface of the photosensitive layer to a thickness of 150 Å, and the hole mobility μ was measured according to the Time of Flight method by irradiating 5-ns pulsed light from a laser of 510 nm. The measurement conditions were selected according to the method described in publications (Philosophical Magazine B, Vol. 58, No. 5, page 539 (1988); J. Phys. Chem., Vol. 88, No. 20, page 4707 (1984)).

Here, it is known that the dependency of the hole mobility μ on the electric field intensity can be quantitatively adjusted according to the following formula. In mathematical formula 1, μ₀ represents the zero field mobility, β represents the Pool-Frenkel parameter, and E represents the electric field intensity,

$$μ = {μ}_{0} {e}^{β \sqrt{Ε}} \qquad ( Mathematical Formula 1 )$$

### Examples 2 to 21

### Measurement of Mobility

Using other compounds of the present invention, photosensitive layers were produced by the same operation as in Example 1, and mobility was measured under the same measurement conditions.

The hole mobility μ under the condition of E=5×10⁴ V/cm determined from the measurement conditions above, the value, and the zero field mobility β₀ calculated according to mathematical formula 1 in each of Examples 1 to 21 are shown in Table 2.

TABLE 2

| | | | | μ (cm²V⁻¹S⁻¹) at |
|---|---|---|---|---|
| | Compound | μ₀ | β | E = 5 × 10⁴ |
| Example | No. | (cm²V⁻¹S⁻¹) | (cm/V)^{0.5} | (V/cm) |
| 1 | 1-2 | 13.5 × 10⁻⁶ | 1.119 × 10⁻³ | 17.3 × 10⁻⁶ |
| 2 | 1-1 | 12.1 × 10⁻⁶ | 1.152 × 10⁻³ | 15.7 × 10⁻⁶ |
| 3 | 1-4 | 11.3 × 10⁻⁶ | 1.176 × 10⁻³ | 14.7 × 10⁻⁶ |
| 4 | 1-6 | 11.9 × 10⁻⁶ | 1.166 × 10⁻³ | 15.4 × 10⁻⁶ |
| 5 | 1-8 | 12.5 × 10⁻⁶ | 1.198 × 10⁻³ | 16.3 × 10⁻⁶ |
| 6 | 1-13 | 8.5 × 10⁻⁶ | 1.144 × 10⁻³ | 11.0 × 10⁻⁶ |
| 7 | 1-14 | 8.1 × 10⁻⁶ | 1.192 × 10⁻³ | 10.6 × 10⁻⁶ |
| 8 | 1-20 | 10.5 × 10⁻⁶ | 1.266 × 10⁻³ | 13.9 × 10⁻⁶ |
| 9 | 1-24 | 8.8 × 10⁻⁶ | 1.305 × 10⁻³ | 11.8 × 10⁻⁶ |
| 10 | 1-25 | 11.4 × 10⁻⁶ | 1.121 × 10⁻³ | 14.6 × 10⁻⁶ |
| 11 | 1-26 | 7.6 × 10⁻⁶ | 1.137 × 10⁻³ | 9.8 × 10⁻⁶ |
| 12 | 1-30 | 8.5 × 10⁻⁶ | 1.313 × 10⁻³ | 11.4 × 10⁻⁶ |
| 13 | 1-38 | 7.8 × 10⁻⁶ | 1.286 × 10⁻³ | 10.4 × 10⁻⁶ |
| 14 | 1-40 | 7.8 × 10⁻⁶ | 1.249 × 10⁻³ | 10.3 × 10⁻⁶ |
| 15 | 1-41 | 8.6 × 10⁻⁶ | 1.275 × 10⁻³ | 11.5 × 10⁻⁶ |
| 16 | 1-42 | 8.5 × 10⁻⁶ | 1.173 × 10⁻³ | 11.1 × 10⁻⁶ |
| 17 | 1-45 | 7.5 × 10⁻⁶ | 1.263 × 10⁻³ | 9.9 × 10⁻⁶ |
| 18 | 1-47 | 9.7 × 10⁻⁶ | 1.259 × 10⁻³ | 12.8 × 10⁻⁶ |
| 19 | 1-54 | 11.9 × 10⁻⁶ | 1.166 × 10⁻³ | 15.4 × 10⁻⁶ |
| 20 | 1-55 | 8.5 × 10⁻⁶ | 1.250 × 10⁻³ | 11.2 × 10⁻⁶ |
| 21 | 1-65 | 9.6 × 10⁻⁶ | 1.178 × 10⁻³ | 12.5 × 10⁻⁶ |

### Comparative Examples 1 to 16

Using comparative compounds represented by the following formula (X) in place of the compound of the present invention, photosensitive layers were formed by the same method as in Example 1, and the mobility was measured. In formula (X), n1 to n4 each represents 1 or 2 and when n1 to n4 each is 2, R9's, R10's, R11's or R12's may be the same or different. Specific substituents R⁹ to R¹² are shown in Table 3. The hole mobility μ, the β value and the zero field mobility μ₀ of each of Comparative Compounds X-1 to X-16 are shown in Table 3.

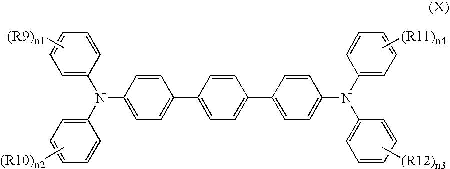

TABLE 3

| | | | | | | | | μ (cm²V⁻¹S⁻¹) |
|---|---|---|---|---|---|---|---|---|
| Comparative | Compound | | | | | μ₀ | β | at E = 5 × 10⁴ |
| Example | No. | R9 | R10 | R11 | R12 | (cm²V⁻¹S⁻¹) | (cm/V)^{0.5} | (V/cm) |
| 1 | X-1 | 3-Me | H | 3-Me | H | 2.4 × 10⁻⁶ | 2.143 × 10⁻³ | 3.9 × 10⁻⁶ |
| 2 | X-2 | 2-Me | 4-n-Pr | 2-Me | 4-n-Pr | 1.7 × 10⁻⁶ | 2.129 × 10⁻³ | 2.7 × 10⁻⁶ |
| 3 | X-3 | 3-Me | 4-styryl | 3-Me | 4-styryl | 3.2 × 10⁻⁶ | 1.905 × 10⁻³ | 4.9 × 10⁻⁶ |
| 4 | X-4 | 2-Me | 4-benzyl | 2-Me | 4-benzyl | 2.8 × 10⁻⁶ | 1.959 × 10⁻³ | 4.3 × 10⁻⁶ |
| 5 | X-5 | 3-Me | 4-n-Bu | 3-Me | 4-n-Bu | 4.3 × 10⁻⁶ | 1.778 × 10⁻³ | 6.4 × 10⁻⁶ |
| 6 | X-6 | 2-Me | 4-n-Bu | 2-Me | 4-n-Bu | 1.6 × 10⁻⁶ | 2.305 × 10⁻³ | 2.7 × 10⁻⁶ |
| | | 5-Me | | 5-Me | | | | |
| 7 | X-7 | 4-iso-Pr | 2-Me | 4-iso-Pr | 2-Me | 1.2 × 10⁻⁶ | 2.159 × 10⁻³ | 1.9 × 10⁻⁶ |
| 8 | X-8 | 4-tert-Bu | 3-Me | 4-tert-Bu | 3-Me | 2.2 × 10⁻⁶ | 2.331 × 10⁻³ | 3.7 × 10⁻⁶ |
| 9 | X-9 | 3-Me | 4-cyclohexyl | 3-Me | 4-cyclohexyl | 1.9 × 10⁻⁶ | 2.373 × 10⁻³ | 3.2 × 10⁻⁶ |
| | | 4-Me | | 4-Me | | | | |
| 10 | X-10 | 2-Me | 4-n-Bu | 2-Me | 4-n-Bu | 4.2 × 10⁻⁶ | 1.910 × 10⁻³ | 6.4 × 10⁻⁶ |
| | | 6-Et | | 6-Et | | | | |
| 11 | X-11 | 4-sec-Pr | 2-Me | 4-sec-Pr | 2-Me | 1.1 × 10⁻⁶ | 2.394 × 10⁻³ | 1.9 × 10⁻⁶ |
| 12 | X-12 | 4-iso-pentyl | H | 4-iso-pentyl | H | 0.9 × 10⁻⁶ | 2.451 × 10⁻³ | 1.6 × 10⁻⁶ |
| 13 | X-13 | 3-Me | 3-Me | 4-n-Pr | 4-n-Pr | 1.8 × 10⁻⁶ | 2.002 × 10⁻³ | 2.8 × 10⁻⁶ |
| 14 | X-14 | 3-Me | 3-Me | 4-n-Bu | 4-n-Bu | 2.6 × 10⁻⁶ | 2.347 × 10⁻³ | 4.4 × 10⁻⁶ |
| | | 4-Me | 4-Me | | | | | |
| 15 | X-15 | 4-iso-Pr | 4-iso-Pr | 3-Me | 3-Me | 2.0 × 10⁻⁶ | 2.316 × 10⁻³ | 3.4 × 10⁻⁶ |
| | | | | 5-Me | 5-Me | | | |
| 16 | X-16 | 4-n-Bu | 4-n-Bu | 2-Ph | 2-Ph | 3.5 × 10⁻⁶ | 1.736 × 10⁻³ | 5.2 × 10⁻⁶ |

### Comparative Examples 17 to 22

Using the following hole transporting compounds in place of the compound of the present invention, photosensitive layers were formed by the same method as in Example 1, and the mobility was measured. The hole mobility μ, the β value and the zero field mobility μ₀ of each of Comparative Examples 17 to 22 and Example 1 are shown in Table 4.

TABLE 4

| | Hole | | | μ |
|---|---|---|---|---|
| | Transport | | | (cm²V⁻¹S⁻¹) at |
| Comparative | Compound | μ₀ | β | E = 5 × 10⁴ |
| Example | No. | (cm²V⁻¹S⁻¹) | (cm/V)^{0.5} | (V/cm) |
| 17 | I-5 | 0.5 × 10⁻⁶ | 2.535 × 10⁻³ | 0.9 × 10⁻⁶ |
| 18 | I-7 | 1.4 × 10⁻⁶ | 2.092 × 10⁻³ | 2.2 × 10⁻⁶ |
| 19 | I-9 | 0.8 × 10⁻⁶ | 2.118 × 10⁻³ | 1.3 × 10⁻⁶ |
| 20 | I-14 | 1.2 × 10⁻⁶ | 2.264 × 10⁻³ | 2.0 × 10⁻⁶ |
| 21 | I-24 | 1.3 × 10⁻⁶ | 2.172 × 10⁻³ | 2.1 × 10⁻⁶ |
| 22 | I-25 | 5.6 × 10⁻⁶ | 1.761 × 10⁻³ | 8.3 × 10⁻⁶ |
| Example 1 | I-2 | 13.5 × 10⁻⁶ | 1.119 × 10⁻³ | 17.3 × 10⁻⁶ |

The zero field mobility μ₀ calculated according to mathematical formula 1 means the hole mobility not including charge migration behavior under high electric field. As seen from the results above, among terphenyl-based hole transport materials known to have high mobility, the compound in a limited structure of the present invention has remarkably high mobility. It is also apparent that the compound of the present invention has significantly higher mobility than conventional hole transport materials and is excellent as a next-generation electrophotographic photoreceptor material.

### Example 22

### Evaluation of Electrophotographic Properties

### <Preparation of Photoreceptor>

1.0 Part of polyvinylbutyral resin (BM-1, produced by Sekisui Chemical Co., Ltd.) was dissolved in 15 parts of methanol, and 5 parts of titanium oxide (TIPAQUE CR-EL, produced by Ishihara Sangyo Kaisha, Ltd.) was added thereto and dispersed in a paint shaker for 2 hours to prepare a coating solution. The obtained coating solution was coated on the aluminum surface of an aluminum-deposited PET film by using a wire bar and dried at 60° C. for 1 hours to form an undercoat layer of 1 μm in thickness. 1.5 Parts of hydroxygallium phthalocyanine having strong peaks at Bragg angles (2θ±0.2°) of 7.5°, 9.9°, 12.5°, 16.3°, 18.6°, 25.1° and 28.3° in the X-ray diffraction spectrum with Cu—Kα was added as a charge generating material to 50 parts of an n-butyl acetate solution of vinyl chloride-vinyl acetate copolymer resin (VMCH, produced by Nippon Unicar Co., Ltd.) and dispersed in a sand mill for 5 hours. The obtained liquid dispersion was coated on the undercoat layer above by using a wire bar and dried at 110° C. for 1 hour to form a charge generating layer having a film thickness of 0.3 μm. Also, 1.5 parts of Compound (1-2) of the present invention was added as a hole transport material to 17 parts of a 10% dichloromethane solution of polycarbonate resin (Iupilon Z, produced by Mitsubishi Engineering-Plastics Corp.) and dissolved, and the resulting solution was coated on the charge generating layer above by using a wire bar and dried at 110° C. for 1 hour to form a charge transport layer having a film thickness of 10 μm, thereby preparing a photoreceptor.

### <Evaluation of Electrophotographic Properties>

The photoreceptor prepared above was subjected to measurement of a photo-induced discharge curve (PIDC) under the following conditions. PIDC shows the relationship between exposure dose and potential on photoreceptor surface and is indicative of device sensitivity. The photoreceptor was negatively charged by corona discharge at −5.0 kV by using an electrostatic copying paper tester (EPA8200, manufactured by Kawaguchi Denki K.K.) in an environment of 20° C. and 50% RH, and halogen lamp light separated to 780 nm was irradiated thereon after adjustment to an intensity of 5.0 μW/cm². The initial surface potential V₀ (V) at this time, the potential-halving exposure dose E_{1/2} (μJ/cm²) required until attenuation of the surface potential to ½ of V₀, and the residual potential V_{R} (V) after 10 seconds from the initiation of exposure are shown in Table 5.

### Examples 22 to 29 and Comparative Examples 23 to 31

Photoreceptors were prepared by the same operation as in Example 22 except for using the compounds shown in Table 5. Subsequently, the electrophotographic properties were evaluated under the same measurement conditions as in Example 22. The results obtained are shown in Table 5.

TABLE 5

| | Compound No. | V₀ (V) | E_{1/2} (μJ/cm²) | V_{R} (V) |
|---|---|---|---|---|
| Example 22 | 1-2 | −486 | 0.13 | −10 |
| Example 23 | 1-1 | −489 | 0.18 | −12 |
| Example 24 | 1-6 | −480 | 0.15 | −14 |
| Example 25 | 1-14 | −477 | 0.33 | −21 |
| Example 26 | 1-20 | −489 | 0.17 | −15 |
| Example 27 | 1-24 | −483 | 0.20 | −17 |
| Example 28 | 1-25 | −486 | 0.23 | −18 |
| Example 29 | 1-42 | −484 | 0.26 | −20 |
| Comparative | X-2 | −487 | 0.58 | −33 |
| Example 23 | | | | |
| Comparative | X-5 | −489 | 0.57 | −37 |
| Example 24 | | | | |
| Comparative | X-9 | −455 | 0.75 | −30 |
| Example 25 | | | | |
| Comparative | X-12 | −462 | 0.62 | −38 |
| Example 26 | | | | |
| Comparative | X-14 | −444 | 0.79 | −30 |
| Example 27 | | | | |
| Comparative | X-16 | −460 | 0.66 | −31 |
| Example 28 | | | | |
| Comparative | I-5 | −460 | 0.98 | −44 |
| Example 29 | | | | |
| Comparative | I-9 | −453 | 1.02 | −47 |
| Example 30 | | | | |
| Comparative | I-25 | −447 | 0.44 | −33 |
| Example 31 | | | | |

It is seen from Table 5 that the photoreceptor using the charge transport material of the present invention is assured of remarkably excellent electrophotographic properties with higher sensitivity than the potential-halving exposure dose E_{1/2} and lower residual potential than V_{R}. The charge transport material of the present invention has excellently high mobility, and the electrophotographic photoreceptor, electrophotographic apparatus or process cartridge using the charge transport material of the present invention can achieve high power and high sensitivity. Accordingly, the chare transport material of the present invention is industrially useful.

According to the present invention, a compound having high charge mobility and being thermally, electrically and chemically stable can be provided. By virtue of this compound, a high-sensitivity, high-durability and high-power electrophotographic photoreceptor ensuring that precipitation of a crystal or production of a pinhole does not occur at the film formation and the film formed as the photosensitive layer is stable, can be provided.

The entire disclosure of each and every foreign patent application from which the benefit of foreign priority has been claimed in the present application is incorporated herein by reference, as if fully set forth.

What is claimed is:

1. An arylamine compound represented by formula (1): 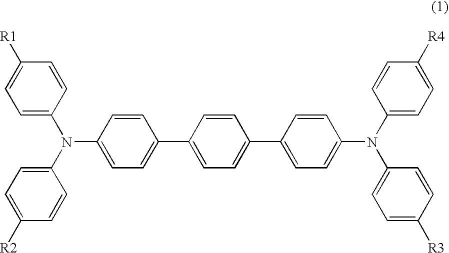
wherein R1 to R4 each independently represents a linear alkyl group or an arylalkenyl group, provided that when all of R1 to R4 represent linear alkyl groups, at least one of R1 to R4 represents a linear alkyl group having a carbon number of 3 or more.

2. The arylamine compound according to claim 1,
wherein R1 to R4 each independently represents a linear alkyl group having a carbon number of 1 to 20 or an alkenyl group having a carbon number of 2 to 20, which has an aryl group having a carbon number of 6 to 10 as a substituent, provided that when all of R1 to R4 represent linear alkyl groups each having a carbon number of 1 to 20, at least one of R1 to R4 represents a linear alkyl group having a carbon number of 3 or more.

3. The arylamine compound according to claim 2,
wherein R1 to R4 each independently represents a linear alkyl group having a carbon number of 1 to 8 or an arylalkenyl group with an alkenyl chain having a carbon number of 2 to 6, provided that when all of R1 to R4 represent linear alkyl groups each having a carbon number of 1 to 8, at least one of R1 to R4 represents a linear alkyl group having a carbon number of 3 or more.

4. The arylamine compound according to claim 3,
wherein R1 to R4 each independently represents a linear alkyl group having a carbon number of 1 to 6 or a mono- or di-arylalkenyl group with an alkenyl chain having a carbon number of 2 to 6, provided that when all of R1 to R4 represent linear alkyl groups each having a carbon number of 1 to 6, at least one of R1 to R4 represents a linear alkyl group having a carbon number of 3 or more.

5. The arylamine compound according to claim 4,
wherein R1 to R4 each independently represents a linear alkyl group having a carbon number of 1 to 4, and at least one of R1 to R4 represents a linear alkyl group having a carbon number of 3 or more.

6. An electrophotographic photoreceptor, comprising:
an electrically conductive support; and
a photosensitive layer that comprises:
a charge generating material comprising at least one selected from the group consisting of selenium, selenium-tellurium, amorphous silicon, pyrylium salt-based dyes, azulenium-based dyes, cyanine-based dyes, squalium salt-based pigments, phthalocyanine-based pigments, anthanthrone-based pigments, dibenzopyrenequinone-based pigments, pyranthrone-based pigments, indigo-based pigments, quinacridone-based pigments, azo pigments and pyrrolopyrrole-based pigments; and
a charge transport material comprising at least one arylamine compound represented by formula (2): 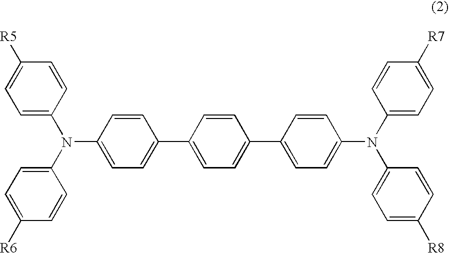
wherein R5 to R8 each independently represents an alkyl group, an arylalkyl group, an aryl group or an arylalkenyl group, provided that R5 to R8 do not represent aryl groups at the same time, and
when all of R5 to R8 represent alkyl groups, or at least one of R5 to R8 represents an alkyl group and the others of R5 to R8 represent aryl groups, at least one of R5 to R8 represents an alkyl group having a carbon number of 3 or more.

7. The electrophotographic photoreceptor according to claim 6,
wherein R5 to R8 each independently represents a linear alkyl group having a carbon number of 1 to 6, and at least one of R5 to R8 represents a linear alkyl group having a carbon number of 3 or more.

8. The electrophotographic photoreceptor according to claim 6,
wherein R5 and R7 are the same and R6 and R8 are the same; or R5 to R8 all are the same.

9. The electrophotographic photoreceptor according to claim 8,
wherein when R5 and R7 are the same and R6 and R8 are the same, a combination of groups represented by R5 and R7, and R6 and R8, is selected from the group consisting of methyl groups and n-propyl groups, methyl groups and n-butyl groups, ethyl groups and n-butyl groups, n-butyl groups and styryl groups, and n-pentyl groups and phenyl groups.

10. The electrophotographic photoreceptor according to claim 6,
wherein the photosensitive layer is a multilayer photosensitive layer that comprises:
a charge generating layer that comprises the charge generating material and has a thickness of 0.01 to 10 μm; and
a charge transport layer that comprises the charge transport material comprising the at least one arylamine compound represented by formula (2) and has a thickness of 1 to 100 μm.
